(19) United States
(12) Patent Application Publication
(10) Pub. No.: US 2007/0255251 A1
(43) Pub. Date: Nov. 1, 2007

(54) Microprojection Array Application With High Barrier Retainer

(75) Inventors: Martin A. Panchula, Vacaville, CA (US); Ling-Kang Tong, Fremont, CA (US)

Correspondence Address: WILSON SONSINI GOODRICH & ROSATI, 650 PAGE MILL ROAD, PALO ALTO, CA, 94304-1050, US

(73) Assignee: ALZA CORPORATION, Mountain View, CA (US)

(21) Appl. No.: 11/684,544

(22) Filed: Mar. 9, 2007

Related U.S. Application Data
(60) Provisional application No. 60/781,049, filed on Mar. 10, 2006.

(51) Int. Cl. A61M 31/00

(52) U.S. Cl. 604/506; 604/174; 604/93.01

(57) ABSTRACT

A transdermal drug delivery system with a high barrier retainer for holding a microprojection member for disrupting a body surface to an individual. The retainer is made of a high barrier material (such as metal) that is easily sterilizable, such as by heat, and can be used for keeping out contamination and maintaining the environment of the microprojection member.

### CROSS-REFERENCE

This application claims the benefit of U.S. Provisional Application No. 60/781,049 filed Mar. 10, 2006, the contents of which are incorporated herein by reference in their entirety.

### TECHNICAL FIELD

This invention relates to an apparatus and method for applying a microprojection member to the stratum corneum by impact, and more particularly, the invention relates to a retainer for mounting a microprojection member having a plurality of microprojections on an impact applicator device to penetrate the stratum corneum with microprojections.

### BACKGROUND

The natural barrier function of the body surface, such as skin, presents a challenge to delivery therapeutics into circulation. Transdermal devices for the delivery of biologically active agents or drugs have been used for maintaining health and therapeutically treating a wide variety of ailments. For example, analgesics, steroids, etc., have been delivered with such devices. Transdermal drug delivery can generally be considered to belong to one of two groups: transport by a “passive” mechanism or by an “active” transport mechanism. In the former, such as drug delivery skin patches, the drug is incorporated in a solid matrix, a reservoir, and/or an adhesive system.

There are various ways to increase transdermal delivery rates. One way to increase the transdermal delivery of agents is to pretreat the skin with, or co-delivering with the beneficial agent, a skin permeation enhancer. A permeation enhancer substance, when applied to a body surface through which the agent is delivered, enhances the transdermal flux of the agent such as by increasing the selectivity and/or permeability of the body surface, and/or reducing the degradation of the agent.

Another type of transdermal drug delivery is active transport in which the drug flux is driven by various forms of energy. Iontophoresis, for example, is an “active” electrotransport delivery technique that transports solubilized drugs across the skin by an electrical current. The feasibility of this mechanism is constrained by the solubility, diffusion and stability of the drugs, as well as electrochemistry in the device. The transport of the agent is induced or enhanced by the application of an applied electrical potential, which results in the application of electric current, to deliver or enhance delivery of the agent.

However, at the present many drugs and pharmaceutical agents still cannot be efficiently delivered by conventional passive patches or electrotransport systems through intact body surfaces. There is an interest in the percutaneous or transdermal delivery of larger molecules such as peptides and proteins to the human body considering the increasing number of medically useful peptides and proteins becoming available in large quantities and pure form. The transdermal delivery of larger molecules such as peptides and proteins still faces significant challenges. In many instances, the rate of delivery or flux of polypeptides through the skin is insufficient to produce a desired therapeutic effect due to their large size and molecular weight. In addition, polypeptides and proteins are easily degraded during and after penetration into the skin, prior to reaching target cells. On the other hand, the passive transdermal flux of many low molecular weight compounds is too limited to be therapeutically effective.

Yet another method to increase transdermal flux (e.g., across skin) is to mechanically penetrate or disrupt the skin. This technique has been mentioned in, for example, U.S. Pat. No. 5,879,326 issued to Godshall, et al., U.S. Pat. No. 3,814,097 issued to Ganderton, et al., U.S. Pat. No. 5,279,544 issued to Gross, et al., U.S. Pat. No. 5,250,023 issued to Lee, et al., U.S. Pat. No. 3,964,482 issued to Gerstel, et al., Reissue 25,637 issued to Kravitz, et al., and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365. These devices use piercing elements or microprojections of various shapes and sizes to pierce the outermost layer (i.e., the stratum corneum) of the skin. The microprojections disclosed in these references generally extend perpendicularly from a thin, flat member, such as a pad or sheet. The microprojections in some of these devices are extremely small, some having dimensions (i.e., a microblade length and width) of only about 25-400 microns (μ) and a microblade thickness of only about 5-50 μ. Other penetrating elements are hollow needles having diameters of about 10 μ or less and lengths of about 50-100 μ. These tiny stratum corneum piercing/cutting elements are meant to make correspondingly small microslits/microcuts in the stratum corneum for enhanced transdermal agent delivery or transdermal body analyte sampling therethrough. The perforated skin provides improved flux for sustained agent delivery or sampling through the skin. In many instances, the microslits/microcuts in the stratum corneum have a length of less than 150 μ and a width that is substantially smaller than their length.

Many microprojection devices have microprojection arrays. When microprojection arrays are used to improve delivery or sampling of agents through the skin, consistent, complete, and repeatable microprojection penetration is desired. Manual application of a skin patch including microprojections often results in significant variation in puncture depth across the microprojection array. In addition, manual application results in large variations in puncture depth between applications due to the manner in which the user applies the array. The reason is that when an individual pushes the microprojection array on the skin by hand, the push force may be hard to control and may be uneven across the area of the array. Accordingly, it would be desirable to be able to apply a microprojection array to the stratum corneum with a mechanically actuated device that provides microprojection skin piercing penetration in a consistent and repeatable manner.

Typically, microprojection arrays have the form of a thin, flat pad or sheet with a plurality of microprojections extending roughly perpendicular therefrom and are difficult to handle manually. Further, such manual application poses a risk of piercing the skin of the handler's fingers. Even if a mechanically actuated applicator is used to apply the microprojection array to the patient, the microprojection array must still be mounted on the applicator. Further, the microprojection array needs to be protected to prevent injury to the user. Thus, a retainer is used in certain devices with microprojection arrays to hold a microprojection member for connection to a reusable impact applicator device for applying the microprojection member to the stratum corneum. For example, US patent application publications 20050148926 and 20050226922 disclose devices with microprojection arrays and retainers. Said patent application publications are incorporated by reference herein in their entireties.

Such prior retainers, however, are still lacking in ease of manufacturing, convenience of sterilization, maintenance of sterility under normal handling, and maintaining an internal protected environment. What is needed is a retainer that can be easily sterilized, can be handled under normal circumstances and still maintain sterility and internal environment without special packaging. The present invention provides system and methods of making and using such systems in which the retainer can be easily manufactured and sterilized and the retainer can act as its own package to provide an environmental barrier from contamination under normal transportation, use and handling.

### SUMMARY

This invention is related to microprojection systems and methodologies that provide a retainer for holding a microprojection member for application of the microprojection member to the stratum corneum with an impact applicator. The microprojection member includes a plurality of microprojections that penetrate the stratum corneum to improve transport of an agent across the stratum corneum. The microprojection member is protected by a retainer made of a heat resistant material. In another aspect of the present invention, a retainer is made with a high barrier material.

In accordance with one aspect of the present invention, a high barrier retainer for a microprojection member is provided. The retainer has a first end attachable to an impact applicator and a second end configured to contact the stratum corneum. A microprojection member having a plurality of stratum corneum-piercing microprojections is positioned within the retainer. Preferably the microprojection member is positioned within the retainer in such a manner that the microprojections are protected from inadvertent contact by the patient or others (e.g., a medical technician) handling the retainer and/or the applicator. The retainer is made of a material that is a high barrier material and/or a heat resistant material. Preferably, the retainer can be sterilized with heat or with gamma radiation.

In accordance with an additional aspect of the invention, a retainer with a barrier seal at its top and bottom ends protecting a microprojection array in the retainer is provided. The retainer and the top and bottom seals are preferably made with high barrier materials. The microprojection patch includes an array of microprojections. Preferably the microprojection member is positioned within the retainer in such a manner that the microprojections are protected from inadvertent contact by the patient or others (e.g., a medical technician) handling the retainer and/or the applicator.

In a further aspect of the invention, a packaged retainer with microprojection member is provided, forming an assembly. The packaged assembly provides a package that can maintain sterility and internal environment under normal transport, storage and handling environment for a medical device such as transdermal drug delivery device. The retainer body and the top and bottom covers act as packaging to provide an aseptic barrier to protect against both contaminations by microorganisms and unwanted gases such as oxygen. The retainer has a body that is configured to be connected to an impact applicator. The microprojection member is mounted on the retainer body for application to the stratum corneum by impact provided by the impact applicator. The retainer acts as part of the packaging to protect against contamination and requires no additional packing material to surround the retainer to protect it. Thus, the retainer can be disposable after a single use and a new unused retainer can be fitted to the same applicator for another episode of drug delivery.

In accordance with another aspect of the invention, a method of applying a microprojection member to the stratum corneum to facilitate delivery or sampling of an agent through the stratum corneum is provided. The method includes the steps of removing high barrier sealing covers from a high barrier retainer, attaching the retainer to an impact applicator, and applying the microprojection member to the stratum corneum with the impact applicator. In another aspect, a retainer is made with a heat resistant material.

In another aspect, the present invention further provides a method of making a high barrier retainer of a material that is a high barrier and/or heat resistant material. The method may include the steps of selecting the parameters for desired properties to form the retainer, such as gas nonpermeability, high temperature tolerance, and adequate mechanical strength. For example, a metallic material can be selected to form the retainer. A preferred embodiment includes stamping a thin sheet metallic material to form a retainer.

In one aspect, this invention includes combining a high barrier package function with the housing function using a formed metal retainer to deliver a microprojection drug delivery system. The formed metal package/housing containing the microprojection system can be covered (as with a lid) on the top and bottom with a removable membrane that is a high barrier or heat resistant material to complete the container/closure system. The retainer and the drug delivery device can be made by selecting the suitable parameters of material and structure for forming the retainer and device to provide gas barriers and heat sterilizability.

This invention combines the drug delivery device with a high barrier and/or heat sterilizable package, thus providing the required environmental barrier while simplifying the packaging and processing. With a high barrier heat resistant retainer, the following advantages are provided:
1. The need for secondary barrier packaging is eliminated.
2. Sterilization of the retainer can be easier using thermal sterilization rather than radiation or gas sterilization, which require more elaborate equipment and process conditions.
3. The metal part is more easily re-cycled and reduces environmental impact. Although some plastics can be recycled, metal cycling can be done very efficiently. For example, aluminum is easily recycled.
4. With the elimination of a separate packaging containing the retainer, the overall package size is reduced. Further, less waste from packaging is generated.

### BRIEF DESCRIPTION OF THE DRAWINGS

The present invention is illustrated by way of example in embodiments and not limitation in the figures of the accompanying drawings in which like references indicate similar elements. The figures are not shown to scale unless indicated otherwise in the content.

FIG. 1 illustrates a sectional view of an applicator device and retainer with microprojection member system according to the present invention.

FIG. 2 illustrates an isometric view of an applicator device and retainer with microprojection member system according to the present invention.

FIG. 3 illustrates an isometric view in portion of a microprojection member according to the present invention.

FIG. 4 illustrates an isometric schematic view in portion of a retainer with top and bottom covers according to the present invention.

FIG. 5 illustrates an exploded isometric view of a retainer with microprojection member and top and bottom covers according to the present invention.

FIG. 6 illustrates an isometric view of the retainer of FIG. 5 packaged with microprojection member and sealed top and bottom covers (shown as transparent) according to the present invention.

FIG. 7 illustrates an isometric view of a retainer without microprojection member according to the present invention.

FIG. 8 illustrates a side view of the retainer of FIG. 7 according to the present invention

### DETAILED DESCRIPTION

The present invention relates to transdermal delivery of drugs using a microprojection device in which a microprojection array is protected in a retainer that can function both as a retainer that facilitates the application and as a package for protecting the microprojection array.

In describing the present invention, the following terms will be employed, and are defined as indicated below. As used in this specification and the appended claims, the singular forms “a,” “an” and “the” include plural references unless the content clearly dictates otherwise.

As used herein, the term “transdermal” refers to the use of skin, mucosa, and/or other body surfaces as a portal for the administration of drugs by topical application of the drug thereto for passage into the systemic circulation.

“Biologically active agent” is to be construed in its broadest sense to mean any material that is intended to produce some biological, beneficial, therapeutic, or other intended effect, such as enhancing permeation or relief of pain. As used herein, the term “drug” refers to any material that is intended to produce some biological, beneficial, therapeutic, or other intended effect, such as relief of pain, but not agents (such as permeation enhancers) the primary effect of which is to aid in the delivery of another biologically active agent such as the therapeutic agent transdermally.

As used herein, the term “therapeutically effective” refers to the amount of drug or the rate of drug administration needed to produce the desired therapeutic result.

The term “high barrier” means that the material is capable of keeping out substantially all gaseous contamination such as oxygen and carbon dioxide, pollutants such as sulfur dioxide and ozone, and water vapor under normal storage and handling for medical devices such as transdermal drug delivery devices, such that the atmosphere within the sealed retainer is substantially free of such gases for a substantial length of time, e.g., at least 6 months, preferably 1 year or more. Moderate barrier materials allow small quantities of these gases to permeate the container, and low barrier materials allow substantial amounts to permeate.

The terms “microprojections” and “microprotrusions”, as used herein, refer to piercing elements that are adapted to pierce or cut through the stratum corneum into the underlying epidermis layer, or epidermis and dermis layers, of the skin of a living animal, particularly a mammal and more particularly a human.

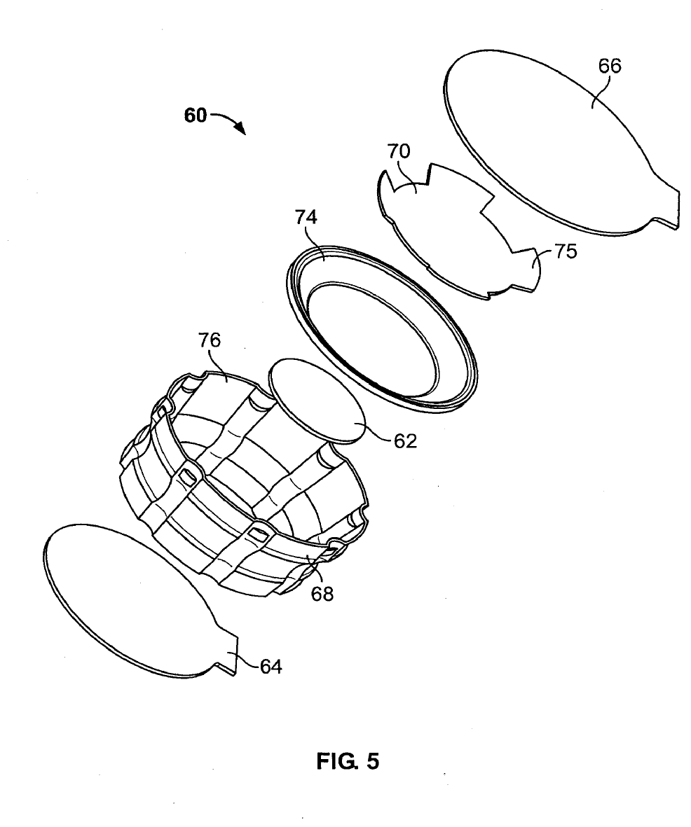

The term “microprojection array” or “microprotrusion array”, as used herein, refers to a plurality of microprojections arranged in an array for piercing the stratum corneum. The microprojection array may be formed by etching or punching a plurality of microprojections from a thin sheet and folding or bending the microprojections out of the plane of the sheet to form a configuration, such as that shown in FIG. 5. The microprojection array may also be formed in other known manners, such as by forming one or more strips having microprojections along an edge of each of the strip(s) as disclosed in U.S. Pat. No. 6,050,988.

In one aspect, the present invention involves methodology that provides a retainer that can function both as a heat resistant retainer for retaining the microprojection array to facilitate application and as a packaging material that protects the mechanical integrity, sterility, and internal environment of the microprojection array in commercial handling during distribution, transport, and storage before the sterility barrier is broken on purpose just prior to deployment on the stratum corneum for beneficial agent or drug delivery. The retainer is used in a microprojection device for transdermal drug delivery. The heat resistant material enables the retainer to be easily heat-sterilized.

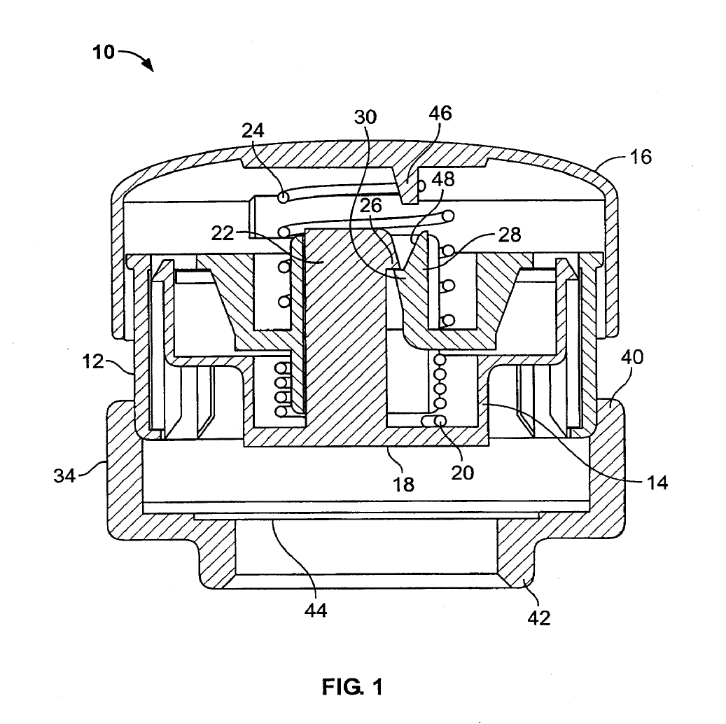

An applicator system for applying a microprojection member as described below includes an impact applicator for applying the microprojection member to the stratum corneum. The microprojection member can include a microprojection array. FIG. 1 shows a schematic sectional view of an exemplary microprojection device that can have a retainer of the present invention. Similar devices with actuators and retainers are described in patent documents 20020123675, 20050096586, 20050138926, 20050226922, and 20050089554, which are incorporated by reference herein. It is understood that such devices of these documents and other prior microprojection devices can be adapted to be used with the retainers of the present invention.

FIG. 1 illustrates an exemplary embodiment of an applicator 10 for use with the retainer 34 of the present invention. However, the device of FIG. 1 is just an example and other applicator configurations may also be used with the retainers described herein. The applicator 10 includes a body 12 and a piston 14 movable within the body. A cap 16 is provided on the body 12 for activating the applicator to impact the stratum corneum with the microprojection member 44. An impact spring 20 is positioned around a post 22 of the piston 14 and biases the piston downward (i.e., towards the skin) with respect to the body 12. The piston 14 has an impact surface 18 that is substantially planar, slightly convex, or configured to match the contours of a particular body surface. The surface 18 of the piston 14 impacts the microprojection member 44 against the skin causing the microprojections 90 to pierce the stratum corneum of, for example, the skin of a patient.

FIG. 1 shows the piston 14 in a cocked position. When the applicator is cocked, the piston 14 is pressed up inside the body 12 and locked in place by a locking mechanism. The locking mechanism includes a stop catch 26 on the post 22 and a flexible finger 28 on the body 12 having a corresponding latch stop 30. As the piston 14 is moved toward the body 12 compressing the impact spring 20, the stop catch 26 flexes the finger 28 and snaps over the corresponding latch stop 30 of the flexible finger. The cocking step is performed by a single compression motion that both cocks and locks the piston 14 in the cocked position.

In the cocked position, catch 26 and latch 30 on the piston 14 and body 12 are releasably engaged, preventing downward motion of the piston in the body. FIG. 1 also illustrates the patch retainer 34 mounted on the body 12. The activation of the applicator 10 by the release of the locking mechanism is performed by downward force applied to the applicator cap 16 while the end 42 of the applicator is held against the skin. The cap 16 is biased in a direction away from the skin by a hold down spring 24 that is positioned between the body 12 and the cap. The cap 16 includes a pin 46 extending downward from the cap. When the cap 16 is pressed downward against the bias of the hold down spring 24, the pin 46 contacts ramp 48 on flexible finger 28 moving the flexible finger outward and disengaging latch 30 of the flexible finger 28 from catch 26. This releases piston 14 and the piston moves downward impacting the stratum corneum with the microprojection member 44. The impact is applied substantially parallel to a central axis of the microprojection member 44. Preferably, the microprojection member is connected to the retainer by at least one frangible element (not shown in the figure) that is broken when the impact applicator is activated.

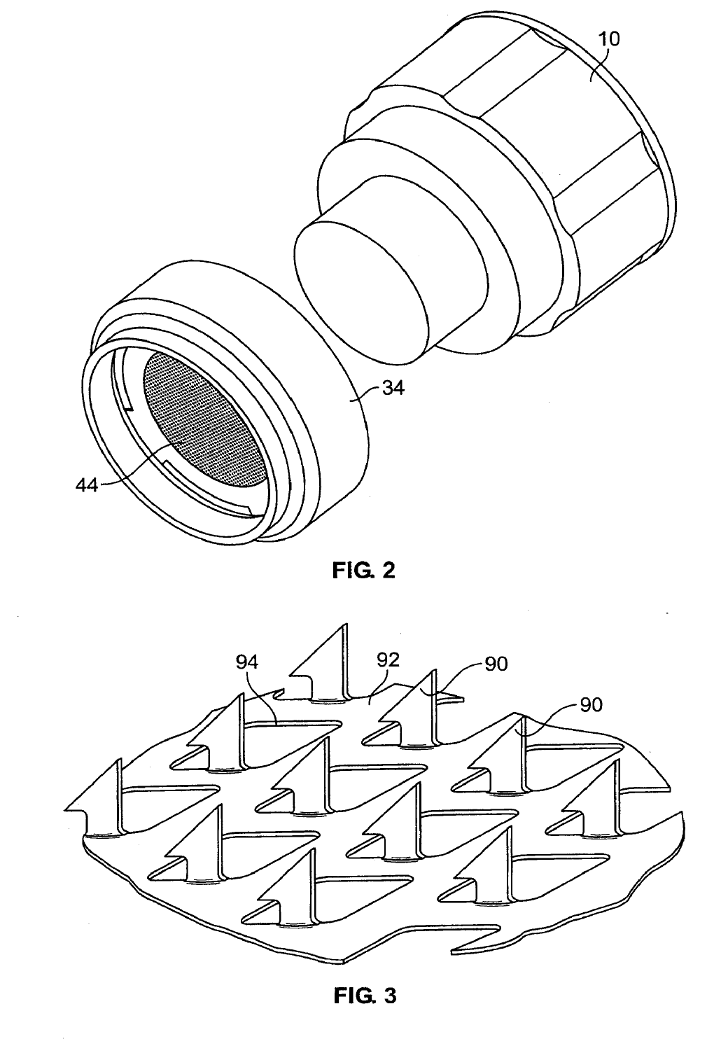

FIG. 2 illustrates an exploded isometric view of an exemplary embodiment of a microprojection device of the present invention. The retainer 34 can be fitted onto the applicator 10. The applicator 10 can thus be used for driving the microprojection member 44 from one end (the end that faces the applicator 10 in this figure) towards the skin through an opening at the other end (the end that is distal to the applicator 10 in this figure). The applicator system of the present invention can be configured to have a reusable impact applicator and a single use microprojection member. In such an embodiment of configuration, the retainer is removably mounted on the impact applicator. After the microprojection member has been applied to (i.e., impacted against) the skin of the patient, the now empty retainer can be removed from the applicator and subsequently a new retainer/microprojection member assembly can be mounted on the applicator. Thus, the retainer is disposable after a single use but the applicator is reusable for application with many retainers. This configuration provides cost benefits since the cost of the applicator can be spread over many microprojection member applications (as opposed to a single application in the case of a single use/completely disposable applicator and retainer and microprojection member assembly.

FIG. 3 illustrates an exemplary embodiment of a microprojection member for use with the present invention. FIG. 3 shows a plurality of microprojections (or microprotrusions) in the form of microblades 90, which have a blade shape with cutting sharp point. The microblades 90 extend at a substantially 90° angle from a sheet 92 having openings 94. The microprojections are preferably sized and shaped to penetrate the stratum corneum of the epidermis when pressure is applied to the microprojection member, for example, forming microslits on the body surface. The sheet 92 may be incorporated in an agent delivery patch or an agent-sampling patch that includes an agent (i.e., a pharmaceutical agent or drug) reservoir and/or an adhesive for adhering the patch to the stratum corneum. Preferably the microprojections each have a drug coating with a drug (for example, on or near the tip of the microprojections). The microprojection member and microprojection array can be made with technology known in the art. Examples of agent delivery and sampling patches that incorporate a microprojection array are found in WO 97/48440, WO 97/48441, WO 97/48442, the disclosures of which are incorporated herein by reference in their entireties. The microprojection array of FIG. 3 without a drug reservoir or a drug coating may also be applied alone as a skin pretreatment.

In one embodiment of the invention, the microprojections have projection length of less than 1000 microns (μ). In a further embodiment, the microprojections have a projection length of less than 500 μ, more preferably, less than 250 μ. The microprojections typically have a width and thickness of about 5 to 50 μ. The microprojections may be formed in different shapes, such as needles, blades, pins, punches, and combinations thereof. The microprojection density is at least approximately 10 microprojections/cm², more preferably, in the range of at least approximately 200-2000 microprojections/cm². The number of openings per unit area through which the active agent (drug) passes is preferably from approximately 10 openings/cm² to about 2000 openings/cm².

The retainer functions for holding and protecting the microprojection member during storage and handling prior to impact against the skin. The retainer is shaped and configured to be mounted on the impact applicator. The retainer has a generally cylindrical (or tubular) shape. As used herein, “cylindrical” or “tubular” means something that has a wall generally ring shaped at an end view with a tunnel in the middle. The “ring” of the ring shaped wall may vary from a perfect circular shape, and thus can be off round, oval, etc., and having undulations thereon. The ring of the ring-shaped wall can also have certain straight portions such as in polygonal shapes (e.g., octagonal). Further, in the cylinder, the diameter need not be longer than the length of the wall but can be shorter in some embodiments. Of course, the tube (or cylinder) can have a diameter and the end of the tube (or cylinder) can be substantially circular shaped (i.e., visually appear to be circular). Even with a substantially circular ring, if desired, there can be undulations along the circumference (and/or along the tubular body) to provide, e.g., grip groove feature, etc.

The retainer allows the microprojection member to be easily loaded on the applicator device without risk of inadvertent contact with the microprojections. The retainer when sealed acts a package to also prevent contamination, folding, or other damage to the microprojection member prior to application. Such a package function eliminates any requirement that an operator use special techniques including hand washing, gloving, sterilized instruments, etc., when handling the retainer with the microprojection member. Although the retainer and microprojection member can be packaged together in an assembled condition as mentioned above, if desired, the retainer can also be packaged separately to provide additional ease of manufacture, sterilization, handling and transportation.

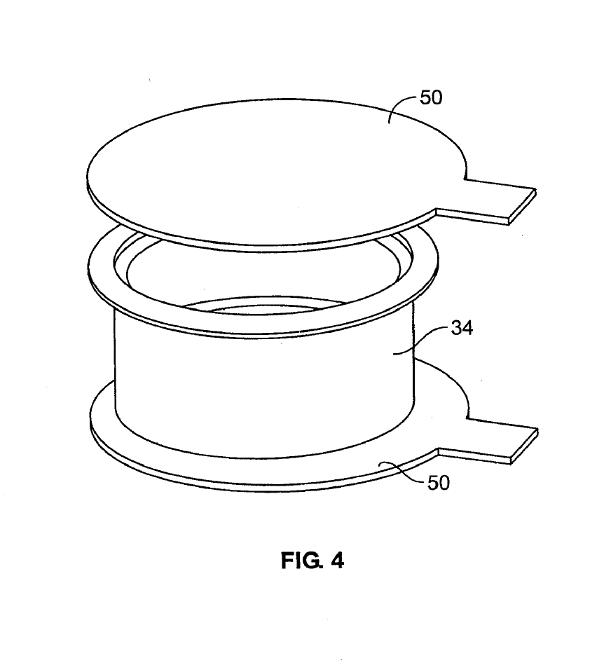

FIG. 4 shows a partially exploded isometric view of an exemplary embodiment in which a retainer 34 is sealed at both ends by sealing covers 50, which aseptically protect the internal structures surrounded by the sealing covers 50 and the exterior wall of the retainer 34. By “aseptically protecting”, it is meant that microorganism contamination such as bacteria and viruses cannot penetrate into the microprojection member inside the retainer under normal handling and storage process in commercial channels and clinical settings such as shipping, transportation, and preparation for application on a patient. It is common knowledge that medical devices are shipped from the manufacturer to various locations (e.g., by trains, trucks, boats, air planes, etc.) for storage (e.g., in the manufacturer's facility, a distributor's facility, hospitals, etc.) and may be handled by a variety of individuals (including shipping personnel, clinical personnel, and patients) under various conditions. Thus, such commercial channels and clinical settings include, e.g., packaging in a manufacturing plant, warehousing, land/sea/air shipping, handling in hospital, clinic, home settings, and other settings known in the art.

Further, the retainer 34 wall and the sealing covers 50 protect the microprojection member from external forces under normal handling and storage process, thus maintaining the mechanical integrity of the microprojection member and other internal structures in the retainer. For example, the retainer and the covers act as the packaging and in all handling process nonsterile parts (such as equipment, fingers, etc.) only contact the exterior of the retainer and the covers. Thus, the mechanical integrity and sterility are not compromised by such shipping and handling activities.

The retainer, for example, that is shown in FIGS. 1, 2 or 4, preferably is made of a material that is heat resistant and heat sterilizable and mechanically strong such that the retainer will not buckle, break or dent under normal shipping, handling, storage and use. Typically, heat sterilization is done with steam at a temperature of 121° C. under a pressure of 15 psi (77.5 cmHg) above atmosphere. Dry heat sterilization is done at 180° C. at atmospheric pressure. If dry heat is employed, the container may be sealed, if steam sterilization is used, the container must remain open during the sterilization process. Further, it is preferred that the retainer is made of a barrier material that can seal against gaseous penetration such as oxygen and water vapor to prevent degradation by mechanisms such as oxidation and hydrolysis.

High barrier materials with respect to pharmaceutical applications generally have water vapor transmission rates of less than 0.155 gm/25 μ/m²/24 hr, and/or oxygen transmission rates less than 1.55 gm/25 μ/m²/24 hr. Moderate barriers can have permeation rates of 0.155-1.55 gm/25 μ/m²/24 hr for water vapor and 1.55-77.5 gm/25 μ/m²/24 hr for oxygen, while low barrier materials having higher corresponding permeation rates. Material for making the retainer would preferably be able to withstand the sterilization temperature and conditions without changing shape during the process. Suitable materials for making a heat resistant and heat sterilizable retainer include metal, ceramic, glass, high barrier polymers such as composites of chloro-fluro polymers, metallized polymers and specialty multi layered structures. Dense materials such as metal, ceramic, glass are preferred because they are impermeable to gases and will not allow gases to pass even after an extremely long period of time, such as years, even decades under normal storage conditions for such medical devices. Such dense materials tend to have specific gravity that is above 2, and for metal, above 3.

Further, these dense materials can stand a much higher temperature than polymeric material, e.g., above 400° C. without softening or changing shape. Such nonpolymeric materials are called “heat-resistant” herein. However, certain polymers filled with nano materials in the dimensional range of less than 1000 nm, such as carbon forms, clays, and various crystalline materials, have been classified as high barrier materials. Such materials can also be used.

Certain polymers (such as polyimides and fluorinated polymers) and adhesives, such as silicone adhesives (siloxane polymer), can tolerate heat sterilization temperatures and can be used in making the device for drug delivery.

For making the retainer, an especially preferred material is metal, which include alloy, such as aluminum, steel, titanium, copper, zinc, tin, and combinations and alloys thereof. Other metals, even precious metal such as gold, silver, and platinum can be use, although such might be costly. Further, other metals or alloys known in the art for making medical device housings can be used. To prevent degradation of the metal or alloy (such as oxidation) that tend to degrade under ordinary atmosphere over time, metal or alloy such as iron, steel, zinc, tin, and the like, would preferably be coated with a protective coating, such as a polymers or anodization. Polymeric and non-organic treatments/coating on metallic materials to prevent oxidation is known in the art.

The retainer containing the microprojection system can also be made as a molded plastic polymeric piece. Preferred polymeric materials that can be used include engineering plastics, which are engineered for structural parts and devices, such as components of machines. Suitable engineering plastics include polypropylene, polycarbonate, and certain high density polyethylenes, and other high temperature materials if thermal sterilization is desired. These are thermoplastics that readily lend themselves to injection molding and extrusion. Other plastics that might be utilized are thermoset plastics such as pheonolics (urea formaldehydes) and the like, and sintered materials such as fluoropolymers that can be compression molded. Ordinary household plastics such as polyethylene, polystyrene, and the like, which are used for consumer household container products, may not provide the high barrier properties (such as oxygen barrier or water vapor barrier) required for the shipping and storage of the microprojection products. Additional outer packaging may be required to provide the required environmental barrier properties.

If desired, a high barrier material can be coated on the plastic material. For example, an ordinary plastic retainer can be coated with a metallic coating, such as aluminum coating. A metallic coating (e.g., aluminum coating) of about 2000 angstroms to 4000 angstroms would be adequate. Thus, when a retainer body is made with a dense (e.g., metallic) material or of a low barrier material coated with a high barrier material (e.g., metallic), the microprojection array is circumferentially encircled around by high barrier material. Further, with such a retainer sealed by higher barrier covers (such as metallic foil or polymeric material with metallic coating), the content of the retainer is completely surrounded by high barrier material on all sides.

FIG. 5 shows an embodiment of a retainer package 60 of the present invention, in which a microprojection member 62 with a microprojection array is packaged aseptically and sealed from contamination by a bottom cover 64 and top cover 66 on a retainer 68. The microprojection array is located on and formed from the microprojection member. The microprojection member is adhesively attached on its side that is opposite from the microblades to an adhesive patch 70 through an opening of an inner retainer (also called backing membrane ring) 74. The adhesive patch 70 in this embodiment is shown to have wings 75 for attaching to the backing membrane ring 74. The adhesive patch is on the back of microprojection member 62 and can be considered as the backing membrane for the microprojection array. The backing membrane ring 74 is held by a slot or a catch 85 inside the retainer 68 for holding the microprojection member 62 (and therefore the microprojection array) inside the retainer 68 until the microprojection member 62 is impacted by the piston of a driver of an applicator when the microprojection array is to be applied to the skin of a patient. The slot 85 is a dent on the inside face of the wall of the retainer corresponding to a longitudinal channel (e.g., grip groove 84).

Preferably, the backing membrane ring 74 is constructed out of a polymeric material, such as polyethylene, polyurethane and polypropylene. In a preferred embodiment, the backing membrane ring 74 is constructed out of polyethylene.

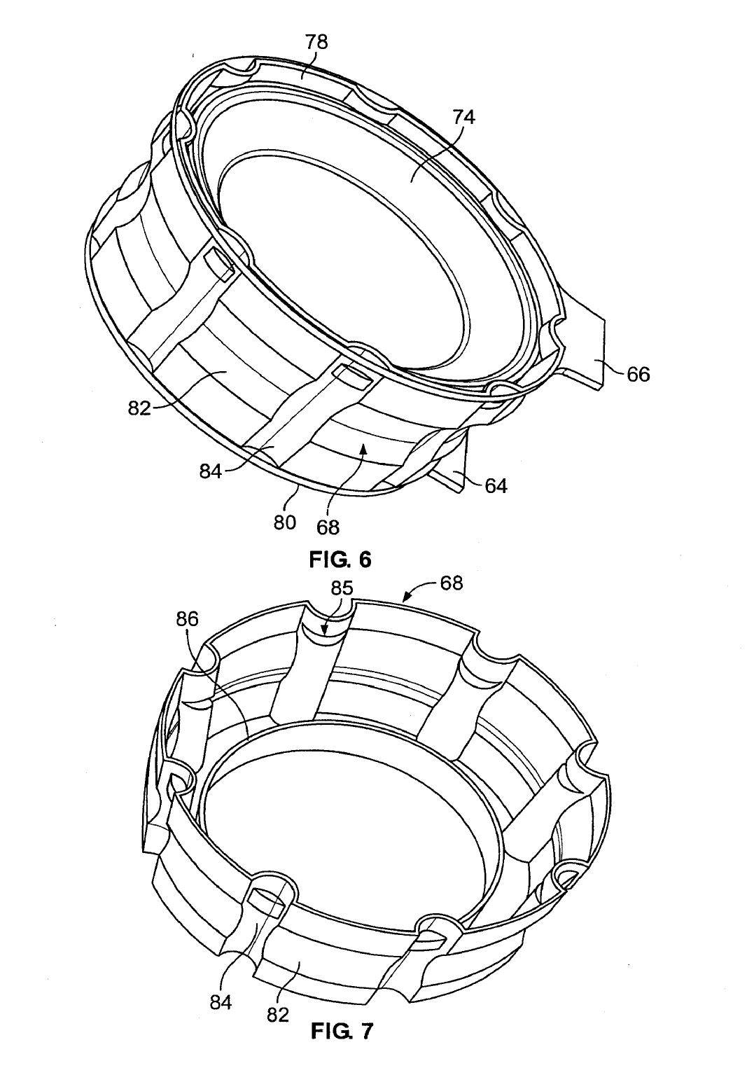

FIG. 6 shows the retainer package 60 in an assembled condition with the top cover 66 and bottom cover 64 sealingly attached to the driver end 78 and exit end 80 of the retainer 68. The top cover 66 is shown as being transparent and in portion for illustration reasons so that the backing membrane ring 74 and adhesive patch 70 can be seen. Before application of the microprojection member 62 with the microprojection array to a patient, the top cover 66 and bottom cover 64 are removed and an applicator is fitted to the retainer 68 with the microprojection member 62 and the backing membrane ring 74 inside the retainer 68.

After an applicator is activated to drive the microprojection member 62, the microprojection member 62 is driven away from the driver end 78 through an opening at the exit end 80 to impact the body surface. The piston of the applicator pushes on the microprojection member 62 and tears the adhesive patch from the backing membrane ring 74 by breaking off at the wings 75. This way, the microprojection member 62 (and with the microprojection array) is kept in sterile condition until use. If desired, the adhesive patch 70 can be made with an adhesive with suitable adhesiveness that the microprojection array will remain on the body surface after the applicator and retainer 68 are pulled away after activation. Suitable adhesives include silicone adhesives such as polydimethylsilosane adhesive (e.g., obtainable from BASF), polyacrylate adhesives such as DUROTAK polyacrylate adhesives (available from National Starch) and polyisobutylene adhesives known in the art. Such applications would be applicable, for example, when a drug has been loaded on the microprojection array for delivery to the tissue under the body surface. The drug will enter the tissue as the microprojection array stays on the skin after impact.

Alternatively, the adhesive patch can be made with a stronger adhesive (i.e., more adhesiveness) such that the microprojection array can be pulled back as well after impacting the body surface. Such an application would be appropriate if a drug is to be applied to the impact site thereafter separate from the microprojection array. Also, it is not necessary that the adhesive patch 70 covers all of the area on the back of the microprojection member 62 (e.g., be a fully disk shape). The adhesive patch can also be attached to the microprojection member 62 only in portion, for example, with an annular shape so that a central area of the microprojection member 62 is not attached to the adhesive patch.

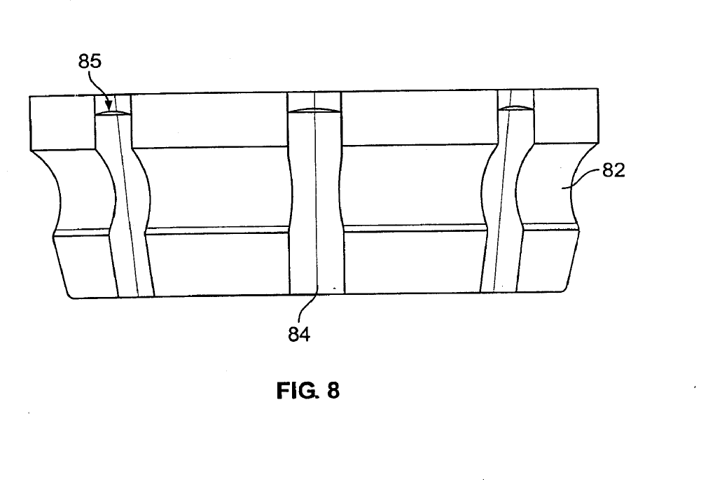

If desired, as shown in FIG. 7 and FIG. 8, to strengthen (i.e., increase) the circumferential stiffness of the retainer 68, the retainer can be made with at least one circumferential channel 82 on the wall of the retainer. Further, to strengthen (i.e., increase) the longitudinal stiffness of the retainer 68, the retainer can be made with one or more longitudinal channels 84. Such channels are useful in increasing the stiffness, particularly in a body having a wall with uniform thickness, for example, as in a retainer made by pressure-forming (press-molding or stamping) a stock piece of material made from a metallic tube, sheet or disks. Mechanical forming processes use die sets, and/or punches to obtain the desired shape from metallic sheets and tubes and are known in the art. Further, such longitudinal channels and circumferential channels (which may be called “grip grooves”) are useful in providing a better grip surface for a more secure finger grip during application. The grip grooves can have, for example, a trough-shape with the cross section of the trough being an arc, a U, or other shapes having an open top and a closed bottom. The grip grooves can have dimensions suitable for fingers to hold the retainer by friction and pressure. For example, the grooves can be from 1 to 10 mm wide and 1 to 10 mm deep. Such dimensions are also convenient for forming the retainer by press molding metallic sheets. A retainer can be made, for example, by forming a long metallic tube first (e.g., by drawing), and sectioning the long metallic tube into sections of appropriate length. Each tube section is a stock piece that can then be processed, i.e., press-molded in a mold to form the shaped retainer with grooves and undulations.

As shown in an embodiment in FIG. 7, the retainer 68 can further have a circular lip 86 on an annular face 85 around the opening at the exit end 80 to further strengthen the circumferential stiffness or the retainer. However, so long as a cover can be affixed to the exit end 80 to seal the content of the retainer, the circular lip 86 and annular face 87, although helpful, are not a necessity. Metallic materials (which may be alloys) are particularly suitable for making retainer with grip grooves and the circular lip because of their ductility and material strength. Many metallic materials (e.g., cupper, aluminum, steel) have sufficient ductility such that they can be fashioned into a new form or shape and retaining the newly formed form or shape under externally applied stress or pressure. Thus, a sheet or a tube of metallic material with substantially uniform sheet or tubular wall thickness by press-molding (or stamping) can form a retainer with grip grooves and retain the new form even under finger grip and actuator operation. The retainer can withstand normal operation with the applicator and finger pressure and retain its mechanical integrity. Such materials are “press-moldable”. Ductility of the metallic material also enables the retainer to have substantially uniform thickness in the retainer wall, in fact, generally in the whole stamped piece of material.

The retainer embodiment as shown in FIG. 7 and FIG. 8 further has slots 85 on the back of all of the longitudinal channels 84 for receiving a backing membrane ring 74 (i.e., inner retainer). The edge of the backing membrane ring 74 can snap into and be held in the slots 85.

To seal the top and bottom ends (i.e., the driver end and the exit end) of the retainer 68, top and bottom covers are provided. Preferably the top and bottom covers are made of barrier material that can protect the microprojection array from microorganism and gaseous (e.g., oxygen) contaminations. Typically, materials suitable for making the top and bottom covers are metal-coated polymeric sheets. For example, sheets of polymeric material such as nylon, polyethylene terephthalate (PET), high density polyethylene bonded to metallic sheet or coated with a metallic film may be used. Metal film bonded high temperature tolerant polymers such as polyimide (e.g., polypyromellitimide), and fluorinated polymers (e.g., polytetrafluoroethylene) can also be used. Metal sheet in thicknesses greater than 10 μ are generally considered high barrier and metallic coatings or 2000 to 4000 angstroms are used to achieve similar results. It is not necessary that the seals and the retainer be made with the same material. It is further not necessary that the top and bottom seals be made with the same material.

There are many ways to seal one or two ends of a tubular container with a cover. For example, sealing mechanisms such as using adhesive, heal seal, stopper, screw caps, crimping, etc. can be used. Such sealing techniques are known in the art.

A particular useful method for sealing a cover sheet on a tubular end is heat sealing, which is easily done and can be applied to a heat-resistant retainer disclosed herein. Further, heat seals are convenient to open. Heat sealing is accomplished by applying heat and pressure to the materials being sealed. In heat sealing, pressure is applied using either a rigid or resilient pad that is heated. The temperature applied is generally between 100° C. and 200° C., but may be more or less, depending on the sealant being used. Adhesives are selected on the basis of the desired heat seal temperature, strength of the resultant bond, and type of materials being sealed together. Simple adhesive systems might be a homopolymer plastic material such as LDPE (low density polyethylene), polypropylene, SURLYN (ionomer), or the like; or may be complex formulation of various resins, waxes, rubbers, as blends. For pharmaceutical applications, for ease of operation thermoplastic polymers can be used. For example, ALCOA SURE-SEAL lidding stock can be used for heat sealing aluminum retainers. Typically, polypropylene sealants are the materials of choice for aluminum retainers, and SURLYN resin for glass retainers. Selection of a heat seal adhesive depends on the specific process and materials selected for use.

When a retainer is sealed with the top and bottom covers, it may be desired that the air or oxygen in the atmosphere under which the device is manufactured is replaced with a sterilized oxygen-free moisture-free gas, such as nitrogen. The process is done with standard techniques to known in the art to maintain the sterility of the device. After the retainer is sealed on top and on bottom, the retainer, with the top and bottom covers, acts as a package to protection against contamination without requiring additional aseptic packaging to preserve sterility of the microprojection member inside the retainer. Of course, in shipping a large quantity of retainers, additional packaging is usually used to transport them as a single unit. For example, boxes made with foam, cardboard, metal, or other material may be used to contain multiple units of retainers. Further, such multiple units and boxes may be shipped in crates, pellets, etc. It is not necessary that such additional packaging be aseptic or sterile since the retainer sealed with top and bottom covers is its own aseptic package.

The retainer with the microprojection member and the top and bottom covers is sterilizable by heat. If sterilized by dry heat, the covers may be in place prior to sterilization. If steam heat or gaseous sterilization is done, the sterilization must be done with one of the covers open and subsequently sealed aseptically. Heat sterilization processes are known in the art. If preferred, sterilization can be done by gamma radiation with the covers in place, with processes known in the art. The covers are removed before the retainer is coupled with an applicator (driver) for applying the microprojection array to the skin.

Pharmaceutically acceptable biologically agents can be put on the microprojection member, e.g., by coating on the microprojections or in the openings in the microprojection member or both. The microprojection member can have one or more pharmaceutically acceptable biologically active agents. For example, the drug coating can have one or more of a variety of drugs or biologically active agents, including traditional pharmaceuticals, as well as small molecules and biologics. Examples of such drugs or biologically active agents include, without limitation, leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotrophin (ACTH), ACTH analogs such as ACTH (1-24), calcitonin, vasopressin, deamino[Val4,D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinin antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-i antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and mixtures thereof.

The drugs or biologically active agents can also be in various forms, such as free bases, acids, charged or uncharged molecules, components of molecular complexes or nonirritating, pharmacologically acceptable salts. Further, simple derivatives of the active agents (such as ethers, esters, amides, etc.), which are easily hydrolyzed at body pH, enzymes, etc., can be employed.

The drugs or biologically active agents can be incorporated into a liquid drug coating material and coated onto the microprojections.

Typically, the drug or biologically active agent is present in the drug coating formulation at a concentration in the range of approximately 0.1-30 wt %, preferably 1-30 wt %. Preferably, the amount of drug contained in the biocompatible coating (i.e., dose) is in the range of approximately 1 μg-1000 μg, more preferably, in the range of approximately 10-200 μg per dosage unit. Even more preferably, the amount of the drug contained in the biocompatible coating is in the range of approximately 10-100 μg per dosage unit.

### EXAMPLES

Below are examples of specific embodiments for carrying out the present invention. The examples are offered for illustrative purposes only, and are not intended to limit the scope of the present invention in any way.

### Example 1

A retainer is made from 200 μ thick anodized aluminum sheet by press molding with longitudinally and circumferentially oriented channels (grip grooves) similar to those shown in FIG. 4. An aluminum sheet is drawn and formed by a sequential punching operation to achieve the desired shape to form a retainer. Microprojection array of about 2 cm diameter with 200 microprojections/cm² is made and placed in the retainer, backed by a backing of polyethylene material with polyacrylate adhesive. Top and bottom covers are made of 25 μ aluminum foil laminated to 25 μ of polyester film for the covers. The cover aluminum foil is coated inside with a sealant and affixed to the retainer by heat sealing. This structure is sterilized with gamma radiation after final cover sealing.

In another embodiment, the retainer is made by press molding a section from a drawn aluminum tube and is sterilized by dry heat at 180° C. at atmospheric pressure for about 2 hours. Heat tolerant polymer material and adhesives are used for the backing material and for the adhesive for the microprojection member.

In another embodiment, the parts are pre-sterilized for aseptic final assembly by gamma, e-beam, dry heat, or steam and other gasses. The parts are then assembled with an aseptic process, under a substantially germ free setting.

The retainers can prevent oxygen and water vapor penetration for a period of more than 6 months, even years.

### Example 2

In a product with the microprojection array that does not demand protection from gaseous environmental factors such as oxygen and moisture, a retainer can be constructed from a barrier plastic. Barrier plastic (polypropylene) is melted and injection molded into a retainer with a nominal wall thickness of 1 mm of polypropylene with grip grooves similar to the shape shown in FIG. 4. Top and bottom covers are made of 25 μ aluminum foil laminated to 25 μ of polyester film for the covers. The cover aluminum foil is coated inside with a sealant ethylene vinyl acetate (EVA) and wax blend (Morprime by Rohm & Haas) and affixed to the retainer by heat sealing or mechanical assembly. This structure is sterilized with gamma radiation after final cover sealing. This results in a moderate environmental barrier. The retainer can prevent oxygen and water vapor penetration for a period of a few days to several months.

### Example 3

Yet another method of construction is to fabricate the retainer with a lower barrier material with high barrier coating. A lower barrier plastic material polystyrene is injection molded into a retainer with 1 mm thick wall and coated with a high barrier material (2000 to 4000 angstroms of aluminum). Similarly, the covers are made of a low barrier material (50 μ polyester film) and coated with a high barrier material (2000 angstrom aluminum) to achieve the desired barrier against gases such as oxygen and moisture. This yields a structure that serves as a high environmental barrier. The retainer can prevent oxygen and water vapor penetration for a period of a few months to years.

The entire disclosure of each patent, patent application, and publication cited or described in this document is hereby incorporated herein by reference. The practice of the present invention will employ, unless otherwise indicated, conventional methods used by those in pharmaceutical product development within those of skill of the art. Embodiments of the present invention have been described with specificity. The embodiments are intended to be illustrative in all respects, rather than restrictive, of the present invention. It is to be understood that various combinations and permutations of various constituents, parts and components of the schemes disclosed herein can be implemented by one skilled in the art without departing from the scope of the present invention.

What is claimed is:

1. Apparatus for stratum-corneum piercing drug delivery, comprising: retainer including a microprojection member having a plurality of stratum corneum piercing microprojections for piercing stratum corneum to deliver a drug, the retainer having a heat resistant tubular body, wherein the microprojection member can be driven from within the retainer towards an end of the tubular body to pierce the stratum corneum.

2. The apparatus of claim 1 wherein the tubular body is gas impermeable including a gas impermeable barrier material and wherein the retainer is disposable.

3. The apparatus of claim 2 wherein the barrier material is selected from the group consisting of metal, ceramic, glass, and high barrier polymer.

4. The apparatus of claim 2 wherein the barrier material is metallic and selected from the group consisting of aluminum, copper, steel, titanium, zinc, tin, and alloys thereof.

5. The apparatus of claim 2 wherein the tubular body can function as aseptic barrier for protecting the sterility and mechanical integrity of the microprojection member during storage and transportation.

6. The apparatus of claim 2 wherein the tubular body has ends on which a heat sterilizable gas impermeable barrier covering can attach for an aseptic seal.

7. The apparatus of claim 2 wherein the tubular body has a wall of uniform thickness.

8. The apparatus of claim 2 wherein the tubular body has a wall of uniform thickness, the wall having one or more longitudinal channel undulatings for improving longitudinal rigidity.

9. The apparatus of claim 2 wherein the tubular body has a wall of uniform thickness, the wall having one or more longitudinal channel and one or more circumferential channel undulatings.

10. The apparatus of claim 2 wherein the apparatus has a size that can be held by fingers for hand held operation.

11. The apparatus of claim 2 further comprising an adhesive surface proximate the microprojection member for adhering to a surface of the stratum corneum to hold the microprojection member thereto when the microprojection member has been driven to pierce the stratum corneum.

12. A method of piercing stratum corneum, comprising:
a. removing an aseptic barrier covering from a retainer to reveal a first opening on the retainer, the retainer having a heat resistant tubular body;
b. fitting an actuator to the first opening of the retainer, and
c. using the actuator to drive a microprojection member having a plurality of stratum corneum piercing microprojections from within the retainer away from said first opening toward the stratum corneum of a patient at a second opening of the retainer for piercing the stratum corneum for drug delivery.

13. The method of claim 12 comprising using a tubular body that is gas impermeable and made from a gas impermeable barrier material and comprising discarding the retainer after use.

14. The method of claim 13 comprising using a tubular body made from a barrier material selected from the group consisting of metal, ceramic, glass, and high barrier polymer.

15. The method of claim 14 comprising using a tubular body made from a metallic material selected from the group consisting of aluminum, copper, steel, titanium, zinc, tin, and alloys thereof.

16. The method of claim 13 comprising using a tubular body that can function as aseptic barrier for protecting the sterility and mechanical integrity of the microprojection member during storage and transportation.

17. The method of claim 13 comprising using a tubular body that has a wall of uniform thickness for protecting the sterility and mechanical integrity of the microprojection member during storage and transportation.

18. The method of claim 13 comprising using a tubular body that has a wall of uniform thickness for protecting the sterility and mechanical integrity of the microprojection member during storage and transportation, the wall having one or more channel undulatings for improving rigidity.

19. A manufacture for use for stratum-corneum piercing drug delivery, comprising: retainer including a microprojection member having a plurality of stratum corneum piercing microprojections for piercing stratum corneum to deliver a drug, the retainer having a heat resistant tubular body, wherein the microprojection member can be driven from within the retainer towards an end of the tubular body to pierce the stratum corneum at the end of the tubular body.

20. The manufacture of claim 19 wherein the tubular body is gas impermeable including a gas impermeable barrier material.

21. The manufacture of claim 20 wherein the barrier material is selected from the group consisting of metal, ceramic, glass, and high barrier polymer.

22. The manufacture of claim 20 wherein the barrier material is metallic and selected from the group consisting of aluminum, copper, steel, titanium, zinc, tin, and alloys thereof.

23. The manufacture of claim 20 wherein the tubular body can function as aseptic barrier when sealed at its ends for protecting the sterility and mechanical integrity of the microprojection member and prevent contamination during storage and transportation.

24. The manufacture of claim 20 further comprising heat sterilizable gas impermeable barrier coverings at two ends of the tubular body forming aseptic seals, wherein said coverings are removable such that the tubular body can receive an actuator at one end for driving the microprojection member to pierce the stratum corneum at another end.

25. A method of making an apparatus for stratum-corneum piercing drug delivery, comprising: coupling a retainer with a driver to form an apparatus for stratum-corneum piercing drug delivery, the retainer including a microprojection member having a plurality of stratum corneum piercing microprojections for piercing stratum corneum to deliver a drug, the retainer having a heat resistant tubular body, wherein the microprojection member can be driven by the driver from within the retainer towards an end of the tubular body to pierce the stratum corneum.

26. The method of claim 25 comprising making the retainer from a heat resistant metallic stock piece of heat resistant material by press-molding the metallic stock piece to form slots therein for holding a member including a microprojection array in a space encircled by the retainer.

27. Use of a biologically active agent together with a carrier microprojection array for treating a subject in need thereof, wherein the microprojection array is held in a retainer, the retainer having a heat resistant tubular body, wherein the biologically active agent is delivered by the microprojection array being driven from within the retainer towards an end of the tubular body to pierce the stratum corneum of the subject.

28. Apparatus for stratum-corneum piercing drug delivery comprising: an applicator comprising a body and a piston movable within the body, a microprojection member having a plurality of stratum corneum piercing microprojections for piercing stratum corneum to deliver a drug; and a cap positioned on the body for activating the applicator to impact the stratum corneum with the microprojection member.

29. The apparatus of claim 28, further comprising an impact spring positioned around a post of the piston, wherein said spring biases the piston with respect to the body.

30. The apparatus of claim 28, wherein the piston has an impact surface that is substantially planar, slightly convex, or configured to match the contours of a particular body surface.

31. The apparatus of claim 30 wherein the surface of the piston impacts the microprojection member against skin of a subject causing the microprojections to pierce the stratum corneum of said subject.

32. The apparatus of claim 28 further comprising a locking mechanism to lock the piston in place inside the body.

33. The apparatus of claim 29 further comprising a locking mechanism to lock the piston in place inside the body.

34. The apparatus of claim 33 wherein the locking mechanism includes a stop catch positioned on a post and a flexible finger positioned on the body, wherein the finger has a corresponding latch stop, whereby when the piston is moved toward the body compressing the impact spring, the stop catch flexes the finger and snaps over the corresponding latch stop of the flexible finger.
